(19) United States
(12) Patent Application Publication
(10) Pub. No.: US 2014/0249380 A1
(43) Pub. Date: Sep. 4, 2014

(54) Methods for Diagnosing Perceived Age Based On An Ensemble Of Phenotypic Features

(71) Applicant: Genocosmetics Lab, SL, Barcelona (ES)

(72) Inventors: José Manuel Mas, Barcelona (ES); Albert Pujol, EI Masnou (ES); Jordi Naval, Barcelona (ES); Raquel Valls, Barcelona (ES); Vicent Carles Alonso, Betera (ES); Miguel Angel Herranz, Barcelona (ES)

(73) Assignee: Genocosmetics Lab, SL, Barcelona (ES)

(21) Appl. No.: 14/176,603

(22) Filed: Feb. 10, 2014

Related U.S. Application Data
(60) Provisional application No. 61/763,190, filed on Feb. 11, 2013.

(51) Int. Cl. A61B 5/00

(52) U.S. Cl. CPC A61B 5/441; A61B 5/4848
USPC 600/301

(57) ABSTRACT

The present invention relates to an algorithm by which phenotypic features of individuals can be calculated and classified to evaluate the overall health of an individual, and particularly skin aging. More particularly, the present invention relates to methods and systems for diagnosing the perceived age of an individual. The invention also relates to methods useful to define products or treatments to reduce the perceived age, to evaluate the efficacy of products and treatments, and to benchmark the product or treatments in order to determine its market value and customer claims.

### BACKGROUND OF THE INVENTION

Biological age of a subject is person's chronological age, whereas perceived age is defined as the age that a person is visually estimated to have, based on their physical appearance. Biological age and perceived age are generally measured in years and parts thereof.

In some people there is a difference between their biological age and their perceived age. The difference between the biological age and perceived age can be a result of various intrinsic and extrinsic factors including but not limited to, exposure to sunlight, pollution, nicotine, and diet or sleeping habits.

Perceived age is a good estimate of health in elderly populations. Perceived age was recently shown to be a clinical marker for assessment of “healthy” aging. Subjects looking old for their age had a greater risk of both morbidity and mortality¹. Also, higher perceived age has been associated with high serum glucose levels², cortisol levels³ and depression state^{4 5}.

Non-genetic factors have a great contribution to perceived age. Changes in facial features, as skin wrinkling⁶, skin color homogeneity^{7,8}, lip size⁶, and sag⁹ have all been linked to perceived age.

The cosmetic industry uses perceived age assessment to determine the efficacy of treatments as per example to quantify the efficacy of multisyringe hyaluronic acid treatment¹⁰ or plastic surgery¹¹.

Perceived age is measured by clinical assessment^{12 13}. Therefore, there is a need for an objective method to determine the perceive age of a person in a faster, better, and more accurate manner.

A method for diagnosing person's perceived age could be applied as an easy and non-invasive method to diagnosis person's health such as waist circumference method as a marker of metabolic syndrome¹⁴ or cardiovascular events¹⁵ and for the evaluation of the efficacy of cosmetic treatments such as contact thermography, morphometric measures of thigh circumference, and microcirculation evaluation used in cellulites¹⁶.

The present invention is directed towards a new, non-obvious and more accurate method for diagnosing perceived age by which phenotypic features of individuals can be measured and classified to evaluate overall health of an individual, and particularly skin ageing.

The method of the present invention is a consistent and standardized method for diagnosing person's perceived age that allows the measurement of a person's perceived age over time and the validation of their treatments which include but not limited to cosmetic treatment, exercise, nutritional complements, diets alternative medicine such as yoga, meditation, relaxation, pilates, laughter therapy, personal growth therapy, psychotherapy or nutritional complements, diets and the similar.

### SUMMARY OF THE INVENTION

The present invention relates to an algorithm by which phenotypic features of individuals can be calculated and classified to evaluate the overall health of an individual, and particularly skin aging. More particularly, the present invention relates to methods and systems for diagnosing the perceived age of an individual.

The invention also relates to methods useful to define products or treatments to reduce the perceived age, to evaluate the efficacy of cosmetic products and treatments, and to benchmark the product in order to determine its market value and customer claims.

The algorithm disclosed in the invention simulates and improves the behavior of clinical assessment performed by an expert panel, determining a person's perceived age with the validated facial grading scale based on individual phenotype criteria given by A. Carruthers¹⁷.

The algorithm of the present invention provides an objective quantification of perceived age, which can be used as a measure to evaluate the overall health of an individual, including but not limited to, skin aging or ageing related disorders associated with the key organs such as liver, lungs, kidney, heart, skin, muscles or bones and biological systems like the central nervous system, digestive, reproduction system and the similar, and more particularly, skin aging.

The invention also provides a diagnostic method based on determining a person's perceived age, useful to design an optimal cosmetic treatment to adjust person's perceived age to their biological age.

The invention also relates to a consistent and standardized diagnostic method that allows measurement of a perceived age over time, thus allowing validation of treatment which include but not limited to cosmetic treatment, exercise, nutritional complements, diets alternative medicine such as yoga, meditation, relaxation, pilates, laughter therapy, personal growth therapy, psychotherapy, and the similar.

The invention also relates to a diagnostic method which determines a person's perceived age, useful to benchmark the product in order to determine its market value and customer claims.

### DETAILED DESCRIPTION OF THE INVENTION

A set of phrases and words to be used in this document are defined in order to avoid uncertainty about the terms.

Biological age: We define the biological age of a subject as a person's chronological age.

Perceived age: We define the perceived age of a subject as the age that a person is visually estimated to have based on their physical appearance.

Biological age and perceived age are generally measured in years and parts thereof. We define the perceived age of a subject as an integer in the range of [−5, +5] years defined by a committee of experts by applying the validated facial grading given by A. Carruthers age [2].

Phenotype: We define phenotype as the set of measurements to take on a subject in terms of their physical appearance.

The present invention relates to an algorithm by which phenotypic features of individuals can be calculated and classified to evaluate the overall health of an individual, and particularly skin aging. More particularly, the present invention relates to methods and systems for diagnosing the perceived age of an individual.

The algorithm disclosed in the invention emulates the behavior of the clinical assessment performed by an expert panel, determining a person's perceived age with the validated facial grading scale based on individual phenotype criteria given by A. Carruthers¹⁷.

Particularly, the invention provides novel methods for developing an algorithm for the diagnosis of a person's perceived age comprising the identification of a set of parameters relevant for determining perceived age, applying an algorithm and obtaining a value that represents the difference between biological age and perceived age.

### Variables Used to Describe the Phenotypic Features of the Skin

A set of parameters known as phenotypic features are defined to develop the algorithm. Two sets of data of phenotypic features of the skin are collected by measuring (1) biophysical parameters derived from ANTERA 3D Miravex device (Dublin, Ireland) or the similar and (2) clinical assessment by an expert committee. Biophysical parameters derived from ANTERA 3D Miravex device include but not limited to wrinkles and roughness, number of wrinkles, depth of wrinkles, pigmentation, concentration of melanin, distribution (heterogeneity) of melanin, superficial vascular component, concentration of hemoglobin, distribution (heterogeneity) of hemoglobin, facial furrows, nasogenian furrow, labiomental groove, roughness and the similar.

In a particular embodiment, the estimation of the concentration of melanin was assessed in the jaw-cheek area of the face by using the biophysical parameters; a) the concentration of melanin, b) the index of variation-heterogeneity of melanin distribution, c) the relative percentage variation and d) the distribution method of the melanin in the area of study in the face.

In a particular embodiment, the estimation of the concentration of hemoglobin was assessed in the jaw-cheek area of the face by using the biophysical parameters: a) the average value of hemoglobin, b) the index of variation-heterogeneity of hemoglobin distribution, c) the relative percentage variation, and d) the distribution method of the hemoglobin in the area of study in the face.

In a particular embodiment, the roughness index of the skin was assessed to estimate the number and intensity of wrinkles by measuring the length and depth of wrinkles in nasogenian furrows and the labiomental grooves area.

Clinically assessed parameters are included but not limited to the position of eyebrows, state of periorbital wrinkles, state of facial wrinkles, evaluation of the labiomental groove and the similar.

In a preferred embodiment, the position of eyebrows is evaluated and scored from 0 (youthful and fresh look, and arched eyebrows) to 4 (droopy and almost flat eyebrows with visible folds and tired appearance).

In a preferred embodiment, the forehead wrinkles is quantified in both resting and dynamic position (maximum elevation of the forehead) in the right and left part of the forehead. The forehead wrinkles are scored from 0 (no wrinkles) to 4 (severe wrinkles).

In a preferred embodiment, the labiomental grooves (“Puppet wrinkles”) are evaluated and scored from 0 (no visible folds) to 4 (extremely long and deep folds).

In a preferred embodiment, the periorbital wrinkles (“Crow's feet”) are evaluated at rest and in movement (maximum contraction of the orbicular muscle) in the right and left periorbital area. The periorbital wrinkles are scored from 0 (none) to 4 (severe).

### Assignment of the Perceived Age of Subjects by an Expert Committee

Data on the perceived age of subjects measured by the test of A. Carruthers¹⁷ is taken in several clinical assessments.

In a preferred embodiment, a set of photos of various individuals is taken in a standardized manner. Photographs are assessed blindly and independently by a committee of experts who assign the age attributed to each one of the subjects based on their expert opinion and professional experience as well as, by using the validated scale age of the facial age gradation suggested by A. Carruthers¹⁷.

In a preferred embodiment, perceived age of a person is defined as an integer in the range of [−5, +5] years defined by a committee of experts upon applying the validated facial grading scale given by A. Carruthers¹⁷. As per example +2 indicates that the subject appears to have two years more than their actual age, and −3 indicates that the subject has the perceived age equivalent of 3 years younger than their actual age.

### Disclosed Algorithm for Diagnosing Perceived Age Based on an Ensemble of Phenotypic Features

The algorithm of the present invention takes phenotype data as variables and expert assignments as objective data.

The method disclosed uses supervised mathematical learning techniques and error retropropagation (backpropagation) for the creation of a neural network (Artificial Neural Network) able to learn biophysical and clinically assessed parameters of subjects.

The typology of the neural network is constructed without a hidden layer, which in statistical terms means that we seek a linear data separator (a line). However, this line separates samples in “n”-dimensional space corresponding to the n phenotype variables.

In a preferred embodiment, the typology of the neural network is constructed without a hidden layer, which in statistical terms means that we seek a linear data separator (a line). However, this line separates samples in “22”-dimensional space corresponding to the 22 phenotype variables.

In a preferred embodiment, the algorithm is inserted into a system that comprise a computer-readable medium; at least one processor coupled with the computer-readable medium; and at least one human-readable output coupled with the computer readable medium and the processor system; wherein the system is capable of executing the algorithm based on an ensemble of phenotypic features of the skin to evaluate overall health of an individual in a specified manner, comprising a database module creating and storing databases of biological data, a first unit operations module transforming the databases into physical features, a second unit operations module generating at least one mathematical model, an analysis module executing experimental analysis and processes, and a comparison module comparing results arising from the models to at least a first set of empirical data.

The algorithm of the present invention is particularly useful to provide an objective quantification of perceived age, which can be used as a measure to evaluate the overall health of one or more individuals, including but not limited to diseases associated to the key organs such as liver, lungs, kidney, heart, skin, muscles, bones and biological systems like the central nervous system, digestive and reproduction systems.

More particularly, the algorithm of the present invention is useful to provide an objective quantification of perceived age, which can be used as a measure to evaluate the overall skin aging of one or more individuals.

The invention also relates to a consistent and standardized diagnostic method that allows measurement of a perceived age over time, thus allowing validation of treatment which include but not limited to cosmetic treatment, alternative medicine, exercise, nutritional complements, diets and the similar.

The phrase “cosmetic treatment” of this invention means a cosmetic products or treatments of biological interest include but not limited to products that have an moisturizing effect, anti-aging, structuring effect, increasing the brightness, thickness and microcirculation, wrinkle fillers, balance the homeostasis of the skin, skin regeneration, dermal metabolism stimulation, skin repair, protection against environmental contamination, revitalizing, improving ionic equilibrium of the skin, energizing for tired skin, balance the pH of the skin, anti-irritation, decreased skin sensitivity, softness, skin conditioning, lifting effect, increases elasticity and firmness, improve eye contour skin barrier, acne redactor, inhibit melanin synthesis and the similar.

The phrase “alternative medicine” of this invention means treatments of biological interest include but not limited to yoga, pilates, meditation, relaxation, laughter therapy, personal growth therapy, psychotherapy, nutritional complements, exercise, ayurvedic medicine, traditional chinese medicine, homeopathy, naturopathy, energy therapies, biofields, electromagnetic fields, mind body therapies, massage, chiropractic, osteopathy and the similar.

The invention also relates to a diagnostic method which determines a person's perceived age, useful to validate the efficacy of cosmetic treatments.

The invention also relates to a diagnostic method which determines a person's perceived age, useful to benchmark the product in order to determine its market value and customer claims.

### EXAMPLE

### Example 1

An example of the practical use of the present invention is described below to clarify all concepts included in the description.

The description is considered sufficient for being applied by those skilled in the art, in the solving of a putative problem.

Epidemiologic noninterventionist cross-sectional study of cases and controls was conducted in 120 female volunteers for the diagnosis of perceived age using the novel algorithm based on an ensemble of skin phenotypic features, described elsewhere.

The epidemiologic study was conducted at the Hospital of Nisa, Valencia (Spain). The study has been approved by the Autonomic Ethics Committee of Clinical Studies of Drugs and Medical Devices of the Valencian Community in Spain (CAEC).

### Criteria for Inclusion in the Study

For inclusion in the study, volunteers had to fulfill the following criteria: women aged between 41 and 49, who attend the dermatology clinic; of all skin types (normal, dry, or oil); who have a predominantly urban lifestyle; non-smokers in the last five years; with no exposure to the sun without protection; no UVA tanning use; absence of severe skin disorders as per example cutaneous carcinoma, melanoma, collagenosis (systemic lupus, scleroderma . . . ), severe acne or rosacea, scars and consequences of other skin diseases; absence of other environmental and lifestyle factors that clinician consider to significantly contribute to attributed perceived age; absence of aesthetic, medical or surgical facial treatments; demonstrating the ability to read and understand all the items in the informed consent document.

The phenotype of all the subjects involved in the study are represented by 22 variables (Table 1)

Variables used to describe the phenotypic features of the skin

Position of eyebrows
State of periorbital wrinkles

Right periorbital zone
Left periorbital zone

State of facial wrinkles

Right frontal zone
Left frontal zone

Evaluation of labiomental groove
Wrinkles and roughness

Length of wrinkles
Thickness of wrinkles
Depth of wrinkles

Pigmentation

Average value of melanin
Variation-heterogeneity index of the melanin distribution
Relative percentage variation
Method of distribution of melanin in the study area

Superficial vascular component

Average value of hemoglobin
Variation-heterogeneity index of the hemoglobin distribution
Relative percentage variation
Method of distribution of hemoglobin in the study area

Depth, Length of the Facial furrows Nasogenian
Depth, Length of the Labiomental groove
Roughness

### Data Collection in the Study:

Data were collected using two measuring methods: biophysical assessment by ANTERA 3D Miravex device¹⁸ and by clinical assessment of the expert panel.

Data Derived from the Using of the ANTERA 3D Miravex Device

Biophysical parameters derived from the using of the ANTERA 3D Miravex device were wrinkles and roughness, number of wrinkles, depth of wrinkles, length of wrinkles, pigmentation, concentration of melanin, distribution (heterogeneity) of melanin, superficial vascular component, concentration of hemoglobin, distribution (heterogeneity) of hemoglobin, facial furrows analysis, nasogenian furrow analysis and roughness.

The estimation of the concentration of melanin was assessed in the jaw-cheek area of the face by using the biophysical parameters; a) the concentration of melanin, b) the index of variation-heterogeneity of melanin distribution, c) the relative percentage variation and d) the distribution method of the melanin in the area of study in the face.

The estimation of the concentration of hemoglobin was assessed in the jaw-cheek area of the face by using the biophysical parameters; a) the average value of hemoglobin, b) the index of variation-heterogeneity of hemoglobin distribution, c) the relative percentage variation, and d) the distribution method of the hemoglobin in the area of study in the face.

The roughness index of the skin was assessed to estimate the number and intensity of wrinkles by measuring the length and depth of wrinkles in nasogenian furrows and labiomental grooves area.

### Data Collected by the Clinical Assessment of an Expert Panel

Several phenotypic features were evaluated by the clinical assessment of an expert panel, following the validated facial grading scale based on individual phenotype criteria given by A. Carruthers¹⁷.

The position of eyebrows was evaluated and scored from 0 (youthful and fresh look and arched eyebrows) to 4 (droopy and almost flat eyebrows with visible folds and tired appearance).

The forehead wrinkles were quantified in resting and dynamic positions (maximum elevation of the forehead) in the right and left part of the forehead. The forehead wrinkles were scored from 0 (no wrinkles) to 4 (severe wrinkles).

The labiomental grooves (“Puppet wrinkles”) were evaluated and scored from 0 (no visible folds) to 4 (extremely long and deep folds).

The periorbital wrinkles (“Crow's feet”) were evaluated at rest and in movement (maximum contraction of the orbicular muscle) in the right and left periorbital area.

The periorbital wrinkles were scored from 0 (none) to 4 (severe).

### Assignment of Perceived Age of Subjects

Data on the perceived age of the subjects measured by the test of A. Carruthers 17 taken in 6 expert assessments.

Specifically, a set of photos of each participant of the study was taken. The photos were taken in a standardized manner, with reproducibility of lighting conditions, photographic settings, and other conditions like no makeup, covered hair, no jewelry. Subsequently, these photographs were assessed blindly and independently by a panel of experts consisting of 6 dermatologists, who assigned an age attributed to each one of the subjects based on their expert opinion and professional experience as well as on the validated scale age of the facial age gradation suggested by A. Carruthers¹⁷.

### Algorithm for Diagnosing Perceived Age Based on an Ensemble of Phenotypic Features

The algorithm of the present invention takes phenotype data as variables and expert assignments as objective data.

The use of supervised mathematical learning techniques and error retropropagation (backpropagation) based on the creation of a neural network (Artificial Neural Network), able to learn from the 120 available cases, is described. The typology of the neural network is constructed without a hidden layer, which in statistical terms means that we seek a linear data separator (a line). However, this line separates samples in a 22-dimensional space corresponding to the 22 phenotype variables.

The contribution of each of the 22 variables derived from measurements by using the ANTERA 3D Miravex device and clinical assessment and the relevance of each variable in the algorithm were calculated (Table 2).

The accuracy is the capability to predict the data outputted from the clinical study. The constructed algorithm is able to correctly classify the samples with the accuracy of 92.04%.

TABLE 3
Effect of the contribution of each of
22 variables involved in the study

| Variables | Relevance |
|---|---|
| Length of Nasogenian groove | 11.67% |
| Variation-heterogeneity index of the hemoglobin | 10.41% |
| distribution | |
| Depth of Nasogenian groove | 10.26% |
| Position of eyebrows | 7.52% |
| Method of distribution of hemoglobin in the study | 6.83% |
| area | |
| Hemoglobin concentration | 6.70% |
| Method of distribution of melanin in the study area | 6.35% |
| Roughness | 6.28% |
| Variation-heterogeneity index of the melanin | 6.25% |
| distribution | |
| % of melanin distribution | 4.56% |
| Length of wrinkles | 4.06% |
| Depth of labiomental groove | 3.71% |
| Thickness of wrinkles | 3.67% |
| Left frontal facial wrinkles | 2.63% |
| Length of Labiomental groove | 2.35% |
| Depth of wrinkles | 1.61% |
| Depth of labiomental groove | 1.38% |
| Right frontal facial wrinkles | 1.26% |
| % of hemoglobin distribution | 1.25% |
| Left perorbital wrinkles | 0.81% |
| Right perorbital wrinkles | 0.33% |
| Melanie concentration | 0.10% |

The algorithm of the invention was able to determine the perceived age of a customer from their phenotypic data with the accuracy of 92%.

### BIBLIOGRAPHY

1. Christensen, K. et al. “Looking old for your age”: genetics and mortality. Epidemiology 15, 251-2 (2004).
2. Noordam, R. et al. High serum glucose levels are associated with a higher perceived age. Age (Dordr) 35, 189-195.
3. Noordam, R. et al. Cortisol serum levels in familial longevity and perceived age: the Leiden longevity study. Psychoneuroendocrinology 37, 1669-75.
4. Rexbye, H. et al. Influence of environmental factors on facial ageing. Age Ageing 35, 110-5 (2006).
5. Tafet, G. E. et al. Correlation between cortisol level and serotonin uptake in patients with chronic stress and depression. Cogn Affect Behav Neurosci 1, 388-93 (2001).
6. Gunn, D. A. et al. Why some women look young for their age. PLoS One 4, e8021 (2009).
7. Matts, P. J., Fink, B., Grammer, K. & Burquest, M. Color homogeneity and visual perception of age, health, and attractiveness of female facial skin. J Am Acad Dermatol 57, 977-84 (2007).
8. Fink, B. et al. Colour homogeneity and visual perception of age, health and attractiveness of male facial skin. J Eur Acad Dermatol Venereol 26, 1486-92.
9. Ozdemir, R. et al. Anatomicohistologic study of the retaining ligaments of the face and use in face lift: retaining ligament correction and SMAS plication. Plast Reconstr Surg 110, 1134-47; discussion 1148-9 (2002).
10. Taub, A. F., Sarnoff, D., Gold, M. & Jacob, C. Effect of multisyringe hyaluronic acid facial rejuvenation on perceived age. Dermatol Surg 36, 322-8.
11. Chauhan, N., Warner, J. P. & Adamson, P. A. Perceived age change after aesthetic facial surgical procedures quantifying outcomes of aging face surgery. Arch Facial Plast Surg 14, 258-62.
12. Guinot, C. et al. Relative contribution of intrinsic vs extrinsic factors to skin aging as determined by a validated skin age score. Arch Dermatol 138, 1454-60 (2002).
13. Bulpitt, C. J., Markowe, H. L. & Shipley, M. J. Why do some people look older than they should?Postgrad Med J 77, 578-81 (2001).
14. Oizumi, T. et al. A proposal for the cutoff point of waist circumference for the diagnosis of metabolic syndrome in the Japanese population. Circ J 70, 1663 (2006).
15. de Koning, L., Merchant, A. T., Pogue, J. & Anand, S. S. Waist circumference and waist-to-hip ratio as predictors of cardiovascular events: meta-regression analysis of prospective studies. Eur Heart J 28, 850-6 (2007).
16. Sparavigna, A., Guglielmini, G., Togni, S., Cristoni. A. & Maramaldi, G. Evaluation of anti-cellulite efficacy: a topical cosmetic treatment for cellulite blemishes—a multifunctional formulation. J Cosmet Sci 62, 305-16.
17. Carruthers, A. & Carruthers, J. A validated facial grading scale: the future of facial ageing measurement tools? J Cosmet Laser Ther 12, 235-41.
18. http://miravex.com/english/antera_{—}3d/antera3d_analysis_of_your_skin-4.html.

1. A method for diagnosing a perceived age of an individual that includes the steps of:
a. measuring phenotypic features of the skin;
b. applying an algorithm; and
c. obtaining a value that represents the difference between biological age and perceived age.

2. A method according to claim 1 where the phenotypic features of the skin are at least 2 features selected from the group consisting in wrinkles and roughness, number of wrinkles, depth of wrinkles, pigmentation, concentration of melanin, distribution (heterogeneity) of melanin, superficial vascular component, concentration of hemoglobin, distribution (heterogeneity) of hemoglobin, facial furrows, nasogenian furrow, labiomental groove, roughness, the position of eyebrows, state of periorbital wrinkles, state of facial wrinkles, evaluation of the labiomental groove.

3. A method according to claim 1 where the algorithm is a neural network-based algorithm with an accuracy value of at least 92%.

4. A method according to claim 3 where all the variables of claim 2 are used in the algorithm.

5. A method of evaluating the health status of an individual comprising using a diagnostic method according to claim 1.

6. A method of assessing a health therapy for an individual in need thereof, comprising using a diagnostic method according to claim 1.

7. A method of assessing a pharmacological therapy for an individual in need thereof, comprising using a diagnostic method according to claim 1.

8. A method of assessing a cosmetic therapy for an individual in need thereof, comprising using a diagnostic method according to claim 1.

9. A method of assessing a cosmetic product for an individual in need thereof, comprising using a diagnostic method according to claim 1.

10. A method of evaluating a treatment efficacy, that comprises the steps of:
evaluating an initial perceived age of an individual using a diagnostic method according to claims 1;
administering a treatment to the individual using the result of the diagnostic method according to claims 1;
for each individual, determine a second perceived age value using a diagnostic method according to claims 1;
for each individual, subtracting first value of perceived age from second value of perceived age; and
associating the difference with the efficacy of the treatment.

11. A system comprising: a computer-readable medium; at least one processor coupled with the computer-readable medium; and at least one human-readable output coupled with the computer readable medium and the processor system; wherein the system is capable of executing the diagnostic method of claim 1 in a specified manner, comprising a database module creating and storing databases of biological and phenotypical data, a first unit operations module transforming the databases into physical features, a second unit operations module executing at least one algorithm, an analysis module executing experimental analysis and processes, a comparison module comparing results arising from the analysis, and an optional output module providing automated interpreted results and assessings.
